(19) United States
(12) Patent Application Publication
(10) Pub. No.: US 2020/0129469 A1
(43) Pub. Date: Apr. 30, 2020

(54) METHODS OF TREATING BILIARY TRACT CANCER

(71) Applicant: Abraxis BioScience, LLC

(72) Inventor: Markus RENSCHLER, Fort Lauderdale, FL (US)

(21) Appl. No.: 16/338,900

(22) PCT Filed: Oct. 6, 2017
(86) PCT No.: PCT/US2017/055559

§ 371 Date: Apr. 2, 2019

Related U.S. Application Data
(60) Provisional application No. 62/405,706, filed on Oct. 7, 2016.

(51) Int. Cl. A61K 31/337; A61K 47/42; A61K 9/00; A61K 9/14; A61P 35/00

(52) U.S. Cl. CPC A61K 31/337; A61K 47/42; A61K 45/06; A61K 9/14; A61P 35/00; A61K 9/0019

(57) ABSTRACT

The present invention provides methods and compositions for treating biliary tract cancers by administering an effective amount of a composition comprising nanoparticles comprising a taxane and an albumin. The present invention also provides combination treatment methods of treating biliary tract cancers comprising administering an effective amount of a composition comprising nanoparticles comprising a taxane and an albumin and an effective amount of another therapeutic agent. Also provided herein are medicines and kits thereof.

This application claims priority from U.S. Provisional Patent Application No. 62/405,706, filed Oct. 7, 2016, the contents of which are incorporated herein by reference in its entirety.

### TECHNICAL FIELD

The present invention provides methods and compositions for treating biliary tract cancers by administering an effective amount of a composition comprising nanoparticles comprising a taxane and an albumin. The present invention also provides combination treatment methods of treating biliary tract cancers comprising administering an effective amount of a composition comprising nanoparticles comprising a taxane and an albumin and an effective amount of another therapeutic agent. Also provided herein are medicines and kits thereof.

### BACKGROUND

Biliary tract cancers are cancers of the bile duct system, a network that transports bile from the liver and gallbladder to the small intestine. The bile duct system starts in the liver with a network of small tubes, called bile canaliculi, which collect bile secreted by hepatocytes. Bile is then transported through the liver through a series of merging ducts, including the Canals of Hering, intrahepatic bile ductules, interlobular bile ducts, and left and right hepatic ducts. The left and right hepatic ducts merge to form the common hepatic duct in an area called the hilum. The cystic duct, which connects to the gallbladder, merges with the common hepatic duct to form the common bile duct. The common bile duct then passes through the pancreas to join with the pancreatic duct, forming the Ampulla of Vater, before connecting with the small intestine.

Biliary tract cancers can be classified by the location of origin. For example, biliary tract cancers that form within the bile duct system of the liver can be referred to as intrahepatic bile duct cancers. Biliary tract cancers that form outside the liver can be referred to as extrahepatic bile duct cancers. Extrahepatic bile duct cancers can further be classified as perihilar (also referred to as hilar) bile duct cancers, which form in the hilum where the left and right hepatic ducts form the common hepatic duct, or distal bile duct cancers. Perihilar bile duct cancers are also commonly referred to as Klatskin tumors.

Biliary tract cancers can also be classified by cell type. A large percentage of all biliary tract cancers are cholangiocarcinomas, most of which are adenocarcinomas. Biliary tract cancers can also be sarcomas, lymphomas, small-cell carcinomas, or squamous cell carcinomas.

First-line treatment, if available as an option, is surgical resection of the biliary tract cancer. If the cancer can be completely removed, surgical resection provides the possibility for a cure of biliary tract cancer. Alternative treatments for biliary tract cancers that cannot be surgical removed include radiotherapy and chemotherapy regimen, such as gemcitabine, cisplatin, fluorouracil, capecitabine, and oxaliplatin, or combinations thereof.

The disclosures of all publications, patents, patent applications, and published patent applications referred to herein are hereby incorporated herein by reference in their entirety.

### BRIEF SUMMARY

The present application in some embodiments provides a method of treating a biliary tract cancer in an individual in need thereof, comprising administering to the individual an effective amount of a composition comprising nanoparticles comprising a taxane and an albumin. In some embodiments, the biliary tract cancer is an intrahepatic bile duct cancer. In some embodiments, the biliary tract cancer is an extrahepatic bile duct cancer. In some embodiments, the extrahepatic bile duct cancer is a perihilar bile duct cancer or a distal bile duct cancer. In some embodiments, the extrahepatic bile duct cancer is Klatskin tumor. In some embodiments, the biliary tract cancer is cholangiocarcinoma. In some embodiments, the biliary tract cancer is adenocarcinoma. In some embodiments, the biliary tract cancer is sarcoma, lymphoma, small-cell carcinoma, or squamous cell carcinoma.

In some embodiments according to any of the methods described above, the biliary tract cancer is early stage biliary tract cancer, non-metastatic biliary tract cancer, primary biliary tract cancer, advanced biliary tract cancer, locally advanced biliary tract cancer, metastatic biliary tract cancer, biliary tract cancer in remission, recurrent biliary tract cancer, biliary tract cancer in an adjuvant setting, or biliary tract cancer in a neoadjuvant setting.

In some embodiments according to any of the methods described above, the method further comprises administering another therapeutic agent. In some embodiments, the method further comprises administering at least one other therapeutic agent. In some embodiments, the other therapeutic agent is an antimetabolite, e.g., gemcitabine. In some embodiments, the other therapeutic agent is a platinum-based agent, e.g., cisplatin. In some embodiments, the other therapeutic agent is a therapeutic antibody. In some embodiments, the nanoparticle composition and the other therapeutic agent are administered simultaneously or sequentially. In some embodiments, the nanoparticle composition and the other therapeutic agent are administered concurrently.

In some embodiments according to any of the methods described above, the composition comprising nanoparticles comprising taxane and albumin is administered intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, hepatic arterial infusion, or by inhalation. In some embodiments, the composition comprising nanoparticles comprising a taxane and albumin is administered intravenously, intraarterially, intrahepatically, or intraportally.

In some embodiments according to any of the methods described above, the method comprises administering another therapeutic agent, wherein the other therapeutic agent is administered intravenously.

In some embodiments according to any of the methods described above, the taxane is paclitaxel.

In some embodiments according to any of the methods described above, the nanoparticles in the composition have an average diameter of less than about 200 nm.

In some embodiments according to any of the methods described above, the taxane in the nanoparticles is coated with albumin.

In some embodiments according to any of the methods described above, the weight ratio of albumin and taxane in the nanoparticle composition is about 1:1 to about 9:1. In some embodiments, the weight ratio of albumin and taxane in the nanoparticle composition is about 9:1.

In some embodiments according to any of the methods described above, the albumin is human albumin.

In some embodiments according to any of the methods described above, the albumin is human serum albumin.

In some embodiments according to any of the methods described above, the individual is human.

The present application in some embodiments provides kits comprising: a) a composition comprising nanoparticles comprising a taxane and an albumin, and b) an instruction for using the nanoparticle composition for treating a biliary tract cancer in an individual. In some embodiments, the kit further comprises another therapeutic agent.

These and other aspects and advantages of the present invention will become apparent from the subsequent detailed description and the appended claims. It is to be understood that one, some, or all of the properties of the various embodiments described herein may be combined to form other embodiments of the present invention.

### DETAILED DESCRIPTION

The present invention provides methods and compositions for treating biliary tract cancers in an individual in need thereof comprising administering to the individual an effective amount of a composition comprising nanoparticles comprising a taxane and an albumin. In some embodiments, there is provided a method of treating a biliary tract cancer in an individual in need thereof comprising administering to the individual an effective amount of a composition comprising nanoparticles comprising paclitaxel and an albumin. In some embodiments, the nanoparticles comprise the taxane associated (e.g., coated) with the albumin. In some embodiments, the average particle size of the nanoparticles in the nanoparticle composition is no more than about 200 nm. In some embodiments, the weight ratio of the albumin and the taxane in the nanoparticle composition is about 9:1. In some embodiments, the albumin is human albumin (such as human serum albumin). In some embodiments, the nanoparticle composition comprises the albumin stabilized nanoparticle formulation of paclitaxel. In some embodiments, the nanoparticle composition is nab-paclitaxel.

In some embodiments, there is provided a method of treating a biliary tract cancer in an individual comprising administering to the individual an effective amount of a composition comprising nanoparticles comprising a taxane and an albumin, wherein the taxane is associated (e.g., coated) with the albumin. In some embodiments, there is provided a method of treating a biliary tract cancer in an individual comprising administering to the individual an effective amount of a composition comprising nanoparticles comprising a taxane and an albumin, wherein the average particle size of the nanoparticles in the nanoparticle composition is no greater than about 200 nm. In some embodiments, there is provided a method of treating a biliary tract cancer in an individual comprising administering to the individual an effective amount of a composition comprising nanoparticles comprising a taxane and an albumin, wherein the taxane is coated with the albumin, and wherein the average particle size of the nanoparticles in the nanoparticle composition is no greater than about 200 nm. In some embodiments, there is provided a method of treating a biliary tract cancer in an individual, comprising administering to the individual an effective amount of a composition comprising nab-paclitaxel.

The present invention also provides methods and compositions for treating biliary tract cancers in an individual in need thereof comprising administering to the individual: a) an effective amount of a composition comprising nanoparticles comprising a taxane and an albumin, and b) an effective amount of another therapeutic agent. In some embodiments, there is provided a method of treating a biliary tract cancer in an individual in need thereof comprising administering to the individual: a) an effective amount of a composition comprising nanoparticles comprising paclitaxel and an albumin, and b) an effective amount of another therapeutic agent. In some embodiments, the nanoparticles comprise the taxane associated (e.g., coated) with the albumin. In some embodiments, the average particle size of the nanoparticles in the nanoparticle composition is no more than about 200 nm. In some embodiments, the weight ratio of the albumin and the taxane in the nanoparticle composition is about 9:1. In some embodiments, the albumin is human albumin (such as human serum albumin). In some embodiments, the nanoparticle composition comprises the albumin stabilized nanoparticle formulation of paclitaxel. In some embodiments, the nanoparticle composition is nab-paclitaxel. In some embodiments, the other therapeutic agent is an antimetabolite, such as gemcitabine. In some embodiments, the other therapeutic agent is a platinum-based agent, such as cisplatin. In some embodiments, the other therapeutic agent is a therapeutic antibody.

In some embodiments, the nanoparticle composition is administered intravenously. In some embodiments, the nanoparticle composition is administered intraportally. In some embodiments, the nanoparticle composition is administered intraarterially. In some embodiments, the nanoparticle composition is administered intraperitoneally. In some embodiments, the nanoparticle composition is administered intrahepatically. In some embodiments, the nanoparticle composition is administered by hepatic arterial infusion. In some embodiments, the nanoparticle composition is administered intravesicularly. In some embodiments, the nanoparticle composition is administered subcutaneously. In some embodiments, the nanoparticle composition is administered intrathecally. In some embodiments, the nanoparticle composition is administered intrapulmonarily. In some embodiments, the nanoparticle composition is administered intramuscularly. In some embodiments, the nanoparticle composition is administered intratracheally. In some embodiments, the nanoparticle composition is administered intraocularly. In some embodiments, the nanoparticle composition is administered transdermally. In some embodiments, the nanoparticle composition is administered orally. In some embodiments, the nanoparticle composition is administered by inhalation.

Biliary tract cancers that can be treated with the methods, kits, and compositions described herein include, but are not limited to, an intrahepatic bile duct cancer, an extrahepatic bile duct cancer, a perihilar bile duct cancer (also known as a hilar bile duct cancer), a distal bile duct cancer, a Klatskin tumor, a cholangiocarcinoma biliary tract cancer, an adenocarcinoma biliary tract cancer, a sarcoma biliary tract cancer, a lymphoma biliary tract cancer, a small-cell carcinoma biliary tract cancer, a squamous cell carcinoma biliary tract cancer. In some embodiments, the biliary tract cancers disclosed herein are an early stage biliary tract cancer, a non-metastatic biliary tract cancer, a primary biliary tract cancer, an advanced biliary tract cancer, a locally advanced biliary tract cancer, a metastatic biliary tract cancer, a biliary tract cancer in remission, a recurrent biliary tract cancer, a biliary tract cancer in an adjuvant setting, and a biliary tract cancer in a neoadjuvant setting.

The methods described herein can be used for any one or more of the following purposes: alleviating one or more symptoms of a biliary tract cancer, delaying progression of a biliary tract cancer, shrinking tumor size in a biliary tract cancer patient, inhibiting tumor growth of a biliary tract cancer, prolonging overall survival, prolonging disease-free survival, prolonging time to disease progression for a biliary tract cancer, preventing or delaying a biliary tract cancer tumor metastasis, reducing a preexisting biliary tract cancer tumor metastasis, reducing incidence or burden of a preexisting biliary tract cancer tumor metastasis, and preventing recurrence of a biliary tract cancer.

Also provided herein are compositions (such as pharmaceutical compositions), medicine, kits, and unit dosages useful for the methods described herein.

### Definitions

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of a pathological consequence of a biliary tract cancer. The methods of the invention contemplate any one or more of these aspects of treatment.

The term “individual” refers to a mammal and includes, but is not limited to, human, bovine, horse, feline, canine, rodent, or primate. In some embodiments, the individual is human.

As used herein, an “at risk” individual is an individual who is at risk of developing a biliary tract cancer. An individual “at risk” may or may not have detectable disease, and may or may not have displayed detectable disease prior to the treatment methods described herein. “At risk” denotes that an individual has one or more so-called risk factors, which are measurable parameters that correlate with development of a biliary tract cancer, which are described herein. An individual having one or more of these risk factors has a higher probability of developing cancer than an individual without these risk factor(s).

“Adjuvant setting” refers to a clinical setting in which an individual has had a history of a biliary tract cancer, and generally (but not necessarily) been responsive to therapy, which includes, but is not limited to, surgery (e.g., surgical resection), radiotherapy, and chemotherapy. However, because of their history of a biliary tract cancer, these individuals are considered at risk of development of the disease. Treatment or administration in the “adjuvant setting” refers to a subsequent mode of treatment. The degree of risk (e.g., when an individual in the adjuvant setting is considered as “high risk” or “low risk”) depends upon several factors, most usually the extent of disease when first treated.

“Neoadjuvant setting” refers to a clinical setting in which the method is carried out before the primary/definitive therapy.

As used herein. “delaying” the development of a biliary tract cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a biliary tract cancer is a method that reduces probability of disease development in a given time frame and/or reduces the extent of the disease in a given time frame, when compared to not using the method. Such comparisons are typically based on clinical studies, using a statistically significant number of subjects. Biliary tract cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to biliary tract cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to a biliary tract cancer, an effective amount comprises an amount sufficient to cause a tumor to shrink and/or to decrease the growth rate of the tumor (such as to suppress tumor growth) or to prevent or delay other unwanted cell proliferation in a biliary tract cancer. In some embodiments, the effective amount is an amount sufficient to delay development of a biliary tract cancer. In some embodiments, the effective amount is an amount sufficient to prevent or delay recurrence. An effective amount can be administered in one or more administrations. In the case of biliary tract cancers, the effective amount of the drug or composition may: (i) reduce the number of epithelioid cells; (ii) reduce tumor size; (iii) inhibit, retard, slow to some extent and preferably stop a biliary tract cancer cell infiltration into peripheral organs: (iv) inhibit (e.g., slow to some extent and preferably stop) tumor metastasis; (v) inhibit tumor growth; (vi) prevent or delay occurrence and/or recurrence of tumor; and/or (vii) relieve to some extent one or more of the symptoms associated with a biliary tract cancer.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, by “combination therapy” or “combination treatments” is meant that a first agent be administered in conjunction with another therapeutic agent, including one or more therapeutic agents. “In conjunction with” refers to administration of one treatment modality in addition to another treatment modality, such as administration of a nanoparticle composition described herein in addition to administration of the other therapeutic agent to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, the term “nab” stands for nanoparticle albumin-bound. For example, nab-paclitaxel is a nanoparticle albumin-bound formulation of paclitaxel.

It is understood that aspects and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X.”

As used herein and in the appended claims, the singular forms “a,” “or,” and “the” include plural referents unless the context clearly dictates otherwise.

### Methods of Treating Biliary Tract Cancers

The invention provides methods of treating a biliary tract cancer in an individual (e.g., human) comprising administering to the individual an effective amount of a composition comprising nanoparticles comprising a taxane and an albumin. It is understood that reference to and description of methods of treating a biliary tract cancer below is exemplary and that this description applies equally to and includes methods of treating a biliary tract cancer using a combination treatment (such administering: a) a composition comprising nanoparticles comprising a taxane and an albumin, and b) another therapeutic agent, or administering: a) a composition comprising nanoparticles comprising a taxane and an albumin, and b) at least one other therapeutic agent).

In some embodiments, the invention provides methods of treating a biliary tract cancer in an individual (e.g., human) comprising administering to the individual an effective amount of a composition comprising nanoparticles comprising a taxane and an albumin. In some embodiments, the invention provides methods of treating a biliary tract cancer in an individual (e.g., human) comprising administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel.

In some embodiments, the biliary tract cancer is an intrahepatic bile duct cancer. In some embodiments, the biliary tract cancer is an extrahepatic bile duct cancer. In some embodiments, the biliary tract cancer is a perihilar bile duct cancer (also known as hilar bile duct cancer). In some embodiments, the biliary tract cancer is a distal bile duct cancer. In some embodiments, the biliary tract cancer is a Klatskin tumor. In some embodiments, the extrahepatic bile duct cancer is a Klatskin tumor. In some embodiments, the biliary tract cancer is cholangiocarcinoma. In some embodiments, the cholangiocarcinoma is adenocarcinoma. In some embodiments, the biliary tract cancer is adenocarcinoma. In some embodiments, the biliary tract cancer is sarcoma. In some embodiments, the biliary tract cancer is lymphoma. In some embodiments, the biliary tract cancer is small-cell carcinoma. In some embodiments, the biliary tract cancer is squamous cell carcinoma.

In some embodiments, the biliary tract cancer is early stage biliary tract cancer, non-metastatic biliary tract cancer, primary biliary tract cancer, advanced biliary tract cancer, locally advanced biliary tract cancer, metastatic biliary tract cancer, biliary tract cancer in remission, or recurrent biliary tract cancer. In some embodiments, the biliary tract cancer is localized resectable (e.g., tumors that are confined to a portion of the liver that allows for complete surgical removal), localized unresectable (e.g., the localized tumors may be unresectable because crucial blood vessel structures are involved), or unresectable (e.g., the tumor has spread to involve other organs. In some embodiments, the biliary tract cancer is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the biliary tract cancer is, according to AJCC (American Joint Commission on Cancer) staging criteria, stage T1, T2, T3, or T4 biliary tract cancer.

The methods provided herein can be used to treat an individual (e.g., human) who has been diagnosed with or is suspected of having a biliary tract cancer. In some embodiments, the individual is human. In some embodiments, the individual is at least about any of 35, 40, 45, 50, 55, 60, 65, 70, 75, 80, or 85 years old. In some embodiments, the individual is of Asian ancestry. In some embodiments, the individual is of American Indian ancestry. In some embodiments, the individual is of Hispanic ancestry. In some embodiments, the individual is male. In some embodiments, the individual is a female. In some embodiments, the individual has a single lesion at presentation. In some embodiments, the individual has multiple lesions at presentation. In some embodiments, the individual is resistant to treatment of biliary tract cancer with other therapeutic agents. In some embodiments, the individual is initially responsive to treatment of biliary tract cancer with other therapeutic agents but has progressed after treatment.

In some embodiments, the individual is a human who exhibits one or more symptoms associated with a biliary tract cancer (e.g., jaundice). In some embodiments, the individual is at an early stage of a biliary tract cancer. In some embodiments, the individual is at an advanced stage of a biliary tract cancer, such as advanced or metastatic biliary tract cancer. In some of embodiments, the individual is genetically or otherwise predisposed (e.g., having a risk factor) to developing a biliary tract cancer. These risk factors include, but are not limited to, age, sex, race, diet, history of previous disease, presence of precursor disease, genetic (e.g., hereditary) considerations, and environmental exposure. In some embodiments, the individuals at risk for a biliary tract cancer include, e.g., those having relatives who have experienced a biliary tract cancer, and those whose risk is determined by analysis of genetic or biochemical markers.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. Any of the methods of treatment provided herein may be used to treat an individual who has not previously been treated. In some embodiments, the method is used as a first line therapy. In some embodiments, the method is used as a second line therapy.

The methods described herein are useful for various aspects of biliary tract cancer treatment. In some embodiments, there is provided a method of inhibiting biliary tract cancer cell proliferation (such as biliary tract cancer tumor growth) in an individual, comprising administering to the individual an effective amount of a composition comprising nanoparticles comprising a taxane and an albumin. In some embodiments, at least about 10% (including for example at least about any of 20%, 30%, 40%, 60%, 70%, 80%, 90%, or 100%) of cell proliferation is inhibited.

In some embodiments, there is provided a method of inhibiting biliary tract cancer tumor metastasis in an individual, comprising administering to the individual an effective amount of a composition comprising nanoparticles comprising a taxane and an albumin. In some embodiments, at least about 10% (including for example at least about any of 20%, 30%, 40%, 60%, 70%, 80%, 90%, or 100%) of metastasis is inhibited. In some embodiments, there is provided a method of inhibiting metastasis to a lymph node. In some embodiments, there is provided a method of inhibiting metastasis to the lung.

In some embodiments, there is provided a method of reducing (such as eradiating) pre-existing biliary tract cancer tumor metastasis (such as pulmonary metastasis or metastasis to the lymph node) in an individual, comprising administering to the individual an effective amount of a composition comprising nanoparticles comprising a taxane and an albumin. In some embodiments, at least about 10% (including for example at least about any of 20%, 30%, 40%, 60%, 70%, 80%, 90%, or 100%) of metastasis is reduced. In some embodiments, there is provided a method of reducing metastasis to a lymph node. In some embodiments, there is provided a method of reducing metastasis to the lung.

In some embodiments, there is provided a method of reducing incidence or burden of pre-existing biliary tract cancer tumor metastasis (such as pulmonary metastasis or metastasis to the lymph node) in an individual, comprising administering to the individual an effective amount of a composition comprising nanoparticles comprising a taxane and an albumin.

In some embodiments, there is provided a method of reducing biliary tract cancer tumor size in an individual, comprising administering to the individual an effective amount of a composition comprising nanoparticles comprising a taxane and an albumin. In some embodiments, the tumor size is reduced at least about 10% (including for example at least about any of 20%, 30%, 40%, 60%, 70%, 80%, 90%, or 100%).

In some embodiments, there is provided a method of prolonging time to disease progression of a biliary tract cancer in an individual, comprising administering to the individual an effective amount of a composition comprising nanoparticles comprising a taxane and an albumin. In some embodiments, the method prolongs the time to disease progression by at least any of 1, 2, 3, 4, 5, 6, 7, 8, 9, 10, 11, or 12 weeks.

In some embodiments, there is provided a method of prolonging survival of an individual having a biliary tract cancer, comprising administering to the individual an effective amount of a composition comprising nanoparticles comprising a taxane and an albumin. In some embodiments, the method prolongs the survival of the individual by at least any of 1, 2, 3, 4, 5, 6, 7, 8, 9, 10, 11, 12, 18, or 24 month.

In some embodiments, there is provided a method of alleviating one or more symptoms in an individual having a biliary tract cancer, comprising administering to the individual an effective amount of a composition comprising nanoparticles comprising a taxane and an albumin.

In some embodiments, there is provided a method of treating biliary tract cancer to obtain an endpoint objective, such as a primary endpoint, secondary endpoint, or exploratory endpoint, including endpoints based on progression free survival (PFS), safety, median time to progression (TIP), overall response rate (ORR), disease control rate (DCR), median progression free survival (PFS), median overall survival (OS), and correlation of change in CA19-9 to clinical efficacy. In some embodiments, the primary endpoint is based on progression free survival, for example, a percentage of a population treated with the methods disclosed herein with progression free survival at a specified time following treatment.

In some embodiments, the method of treating an intrahepatic bile duct cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the intrahepatic bile duct cancer is cholangiocarcinoma. In some embodiments, the intrahepatic bile duct cancer is adenocarcinoma. In some embodiments, the intrahepatic bile duct cancer is sarcoma. In some embodiments, the intrahepatic bile duct cancer is lymphoma. In some embodiments, the intrahepatic bile duct cancer is small-cell carcinoma. In some embodiments, the intrahepatic bile duct cancer is squamous cell carcinoma.

In some embodiments, the method of treating an extrahepatic bile duct cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the extrahepatic bile duct cancer is cholangiocarcinoma. In some embodiments, the extrahepatic bile duct cancer is adenocarcinoma. In some embodiments, the extrahepatic bile duct cancer is sarcoma. In some embodiments, the extrahepatic bile duct cancer is lymphoma. In some embodiments, the extrahepatic bile duct cancer is small-cell carcinoma. In some embodiments, the extrahepatic bile duct cancer is squamous cell carcinoma.

In some embodiments, the method of treating a perihilar bile duct cancer (also known as hilar bile duct cancer) in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the perihilar bile duct cancer is cholangiocarcinoma. In some embodiments, the perihilar bile duct cancer is adenocarcinoma. In some embodiments, the perihilar bile duct cancer is sarcoma. In some embodiments, the perihilar bile duct cancer is lymphoma. In some embodiments, the perihilar bile duct cancer is small-cell carcinoma. In some embodiments, the perihilar bile duct cancer is squamous cell carcinoma.

In some embodiments, the method of treating a distal bile duct cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the distal bile duct cancer is cholangiocarcinoma. In some embodiments, the distal bile duct cancer is adenocarcinoma. In some embodiments, the distal bile duct cancer is sarcoma. In some embodiments, the distal bile duct cancer is lymphoma. In some embodiments, the distal bile duct cancer is small-cell carcinoma. In some embodiments, the distal bile duct cancer is squamous cell carcinoma.

In some embodiments, the method of treating a Klatskin tumor in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating a cholangiocarcinoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating an adenocarcinoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating a sarcoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating a lymphoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating a small-cell carcinoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, the method of treating a squamous cell carcinoma biliary tract cancer in an individual (e.g., human) comprises administering to the individual an effective amount of a composition comprising nanoparticles comprising a taxane and an albumin. In some embodiments, the method comprises administering to the individual an effective amount of a composition comprising nanoparticles comprising paclitaxel and an albumin. In some embodiments, the method comprises administering to the individual an effective amount of a composition comprising nanoparticles comprising a taxane and an albumin, wherein the taxane in the nanoparticles is associated (e.g., coated) with the albumin. In some embodiments, the method comprises administering to the individual an effective amount of a composition comprising nanoparticles comprising a taxane and an albumin, wherein the nanoparticles have an average particle size of no greater than about 200 nm. In some embodiments, the method comprises administering to the individual an effective amount of a composition comprising nanoparticles comprising a taxane and an albumin, wherein the nanoparticles comprise a taxane associated (e.g., coated) with albumin, and wherein the nanoparticles have an average particle size of no greater than about 200 nm. In some embodiments, the method comprises administering to the individual an effective amount of a composition comprising nanoparticles comprising a taxane and an albumin, wherein the weight ratio of albumin and taxane in the nanoparticle composition is about 1:1 to about 9:1. In some embodiments, the method comprises administering to the individual an effective amount of a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1). In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously.

In some embodiments, methods of treating a biliary tract cancer in an individual comprise administering to the individual an effective amount of a composition comprising nanoparticles comprising a taxane and an albumin, wherein the individual is selected for treatment based on the presence of a biomarker. In some embodiments, the individual is selected for treatment based on a low level of a biomarker. In some embodiments, the individual is selected for treatment based on a high level of a biomarker. In some embodiments, the method further comprises selecting the individual based on the presence or level of a biomarker. In some embodiments, the biomarker is selected from the group consisting of cytidine deaminase (CDA), human equilibrative nucleoside transporter 1 (hENT1), and secreted protein acidic and rich in cysteine (SPARC). In some embodiments, the biomarker is a tumor biomarker. In some embodiments, the tumor biomarker is selected from the group consisting of cytidine deaminase (CDA), human equilibrative nucleoside transporter 1 (hENT1), and secreted protein acidic and rich in cysteine (SPARC). In some embodiments, the biomarker is a stromal biomarker. In some embodiments, the stromal biomarker is selected from the group consisting of cytidine deaminase (CDA), human equilibrative nucleoside transporter 1 (hENT1), and secreted protein acidic and rich in cysteine (SPARC).

In some embodiments, the biliary tract cancer is stromal-rich. In some embodiments, the biomarker is the presence of fibrosis. In some embodiments, the biomarker is a high level of fibrosis. In some embodiments, the biomarker is a low level of fibrosis. Fibrosis and the level of fibrosis may be measured by, e.g., immunohistochemistry (IHC), elastography, magnetic resonance, computed tomography, or combinations thereof. In some embodiments, the level of fibrosis is high if the fibrosis IHC staining is about 50%, 55%, 60%, 65%, 70%, 75%, 80%, 85%, 90%, or 95% or more intense than a control sample (e.g., a negative control sample). In some embodiments, the level of fibrosis is low if the IHC staining is about 50%, 45%, 40%, 35%, 30%, 25%, 20%, 15%, 10%, or 5% or less intense than a control sample.

In some embodiments, the biomarker is the presence or level of a circulating tumor cell (CTC). In some embodiments, the biomarker is the presence or level of a gemcitabine metabolite.

In some embodiments, the level of the biomarker is determined (e.g., high or low) by comparing to a control. In some embodiments, the level of the biomarker is determined (e.g., high or low) by comparing to another tissue sample from the individual (e.g., adjacent healthy tissue).

In some embodiments, the level of a biomarker is high if the biomarker in a biliary tract cancer sample or stromal sample of the biliary tract cancer is about 50%, 55%, 60%, 65%, 70%, 75%, 80%, 85%, 90%, or 95% or more than a control sample. In some embodiments, the level of a biomarker is low if the biomarker in a biliary tract cancer sample or stromal sample of the biliary tract cancer is about 50%, 45%, 40%, 35%, 30%, 25%, 20%, 15%, 10%, or 5% or less than a control sample.

It is understood that any of the embodiments described in this section apply to the combination treatments, such as embodiments provided in the section “Methods of Combination Treatments.”

### Methods of Combination Treatments

The present invention also provides methods of administering the composition comprising nanoparticles comprising a taxane and an albumin, wherein, in some embodiments, administering the nanoparticle composition is carried out in conjunction with administering at least one other therapeutic agent. In some embodiments, the taxane nanoparticle composition is administered in conjunction with an antimetabolite, such as gemcitabine, and a platinum-based agent, such as cisplatin. In some embodiments, the method is used as a first-line therapy. In some embodiments, the method is used as a second-line therapy.

In some embodiments, the invention provides methods of treating a biliary tract cancer in an individual (e.g., human) comprising administering to the individual an effective amount of: a) a composition comprising nanoparticles comprising a taxane and an albumin, and b) another therapeutic agent. In some embodiments, the invention provides methods of treating a biliary tract cancer in an individual (e.g., human) comprising administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1, and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the other therapeutic agent is an antimetabolite, such as gemcitabine. In some embodiments, the other therapeutic agent is an antimetabolite, such as cisplatin. In some embodiments, the taxane nanoparticle composition is administered in conjunction with an antimetabolite, such as gemcitabine, and a platinum-based agent, such as cisplatin. In some embodiments, the other therapeutic agent is a therapeutic antibody.

In some embodiments, the biliary tract cancer is an intrahepatic bile duct cancer. In some embodiments, the biliary tract cancer is an extrahepatic bile duct cancer. In some embodiments, the biliary tract cancer is a perihilar bile duct cancer (also known as hilar bile duct cancer). In some embodiments, the biliary tract cancer is a distal bile duct cancer. In some embodiments, the biliary tract cancer is a Klatskin tumor. In some embodiments, the extrahepatic bile duct cancer is a Klatskin tumor. In some embodiments, the biliary tract cancer is cholangiocarcinoma. In some embodiments, the cholangiocarcinoma is adenocarcinoma. In some embodiments, the biliary tract cancer is adenocarcinoma. In some embodiments, the biliary tract cancer is sarcoma. In some embodiments, the biliary tract cancer is lymphoma. In some embodiments, the biliary tract cancer is small-cell carcinoma. In some embodiments, the biliary tract cancer is squamous cell carcinoma.

In some embodiments, the biliary tract cancer is early stage biliary tract cancer, non-metastatic biliary tract cancer, primary biliary tract cancer, advanced biliary tract cancer, locally advanced biliary tract cancer, metastatic biliary tract cancer, biliary tract cancer in remission, or recurrent biliary tract cancer. In some embodiments, the biliary tract cancer is localized resectable (e.g., tumors that are confined to a portion of the liver that allows for complete surgical removal), localized unresectable (e.g., the localized tumors may be unresectable because crucial blood vessel structures are involved), or unresectable (e.g., the tumor has spread to involve other organs. In some embodiments, the biliary tract cancer is, according to TNM classifications, a stage I tumor (single tumor without vascular invasion), a stage II tumor (single tumor with vascular invasion, or multiple tumors, none greater than 5 cm), a stage III tumor (multiple tumors, any greater than 5 cm), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the biliary tract cancer is, according to AJCC (American Joint Commission on Cancer) staging criteria, stage T1, T2, T3, or T4 biliary tract cancer.

In some embodiments, the individual is initially responsive to treatment of biliary tract cancer with other therapeutic agents but has progressed after treatment. In some embodiments, the individual is initially responsive to treatment of biliary tract cancer with the other therapeutic agent but has progressed after treatment. In some embodiments, the individual is non-responsive to treatment of biliary tract cancer with the other therapeutic agent.

In some embodiments, the other therapeutic agent is an antimetabolite. In some embodiments, the other therapeutic agent is a fluoropyrimidine. In some embodiments, the other therapeutic agent is gemcitabine. In some embodiments, the other therapeutic agent is 5-fluorouracil.

In some embodiments, the other therapeutic agent is a platinum-based agent. In some embodiments, the other therapeutic agent is cisplatin. In some embodiments, the other therapeutic agent is carboplatin.

The other therapeutic agents contemplated herein include agents that affect (such as inhibit) signaling pathways (such as ligand-receptor-mediated signaling) involved with tumor progression (such as tumor growth and proliferation and angiogenesis).

In some embodiments, the other therapeutic agent inhibits ligand-receptor binding. For example, the other therapeutic agent binds a ligand to inhibit ligand-receptor binding and/or ligand-receptor-mediated signaling.

In some embodiments, the other therapeutic agent is a therapeutic antibody. In some embodiments, the therapeutic antibody binds a ligand for a receptor. In some embodiments, the therapeutic antibody binds a ligand to inhibit ligand-receptor binding. In some embodiments, the therapeutic antibody binds a ligand to inhibit ligand-receptor-mediated signaling. In some embodiments, the therapeutic antibody is an anti-ligand antibody. In some embodiments, the therapeutic antibody is an anti-receptor antibody.

In some embodiments, the other therapeutic agent is an epidermal growth factor receptor inhibitor. In some embodiments, the other therapeutic agent is an anti-epidermal growth factor receptor (EGFR) agent. In some embodiments, the anti-EGFR agent is an anti-EGFR antibody.

In some embodiments, the other therapeutic agent is an anti-angiogenesis agent. Anti-angiogenesis agents contemplated herein include agents that inhibit formation of new vasculature and agents that lead to formation of non-functional vasculature. In some embodiments, the therapeutic antibody is an anti-angiogenesis agent. In some embodiments, the anti-angiogenesis agent binds to vascular endothelial growth factor (VEGF). In some embodiments, the anti-angiogenesis agent binds to vascular endothelial growth factor (VEGF), wherein VEGF-receptor binding is inhibited. In some embodiments, the anti-angiogenesis agent is an anti-VEGF agent (such as an anti-VEGF antibody). In some embodiments, the anti-angiogenesis agent is an anti-angiogenic receptor agent. In some embodiments, the anti-angiogenesis agent is a VEGFR antibody. In some embodiments, the anti-angiogenesis agent is an anti-Notch receptor agent. In some embodiments, the anti-angiogenesis agent is an anti-Notch receptor antibody. In some embodiments, the anti-angiogenesis agent is an anti-Notch ligand agent. In some embodiments, the anti-angiogenesis agent is an anti-Notch ligand antibody.

In some embodiments, the other therapeutic agent is a Wnt pathway inhibitor. In some embodiments, the other therapeutic agent is an anti-Wnt3a antibody. In some embodiments, the other therapeutic agent is an anti-frizzled receptor antibody.

In some embodiments, the other therapeutic agent is administered in conjunction with a third agent or radiation therapy.

In some embodiments, a lower amount of each pharmaceutically active compound is used as part of a combination treatment compared to the amount generally used for individual therapy. In some embodiments, the same or greater therapeutic benefit is achieved using a combination treatment than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. For example, the use of a small amount of pharmaceutically active compound may result in a reduction in the number, severity, frequency, or duration of one or more side-effects associated with the compound.

In some embodiments, the nanoparticle composition and the other therapeutic agent have synergistic effect on treating a biliary tract cancer. In some embodiments, the other therapeutic agent sensitizes the biliary tract cancer cells to the treatment with the nanoparticle composition. In some embodiments, the nanoparticle composition sensitizes the biliary tract cancer cells to the treatment with the other therapeutic agent.

In some embodiments, the method of treating an intrahepatic bile duct cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously. In some embodiments, the intrahepatic bile duct cancer is cholangiocarcinoma. In some embodiments, the intrahepatic bile duct cancer is adenocarcinoma. In some embodiments, the intrahepatic bile duct cancer is sarcoma. In some embodiments, the intrahepatic bile duct cancer is lymphoma. In some embodiments, the intrahepatic bile duct cancer is small-cell carcinoma. In some embodiments, the intrahepatic bile duct cancer is squamous cell carcinoma.

In some embodiments, the method of treating an extrahepatic bile duct cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously. In some embodiments, the extrahepatic bile duct cancer is cholangiocarcinoma. In some embodiments, the extrahepatic bile duct cancer is adenocarcinoma. In some embodiments, the extrahepatic bile duct cancer is sarcoma. In some embodiments, the extrahepatic bile duct cancer is lymphoma. In some embodiments, the extrahepatic bile duct cancer is small-cell carcinoma. In some embodiments, the extrahepatic bile duct cancer is squamous cell carcinoma.

In some embodiments, the method of treating a perihilar bile duct cancer (also known as a hilar bile duct cancer) in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously. In some embodiments, the perihilar bile duct cancer (also known as a hilar bile duct cancer) is cholangiocarcinoma. In some embodiments, the perihilar bile duct cancer (also known as a hilar bile duct cancer) is adenocarcinoma. In some embodiments, the perihilar bile duct cancer (also known as a hilar bile duct cancer) is sarcoma. In some embodiments, the perihilar bile duct cancer (also known as a hilar bile duct cancer) is lymphoma. In some embodiments, the perihilar bile duct cancer (also known as a hilar bile duct cancer) is small-cell carcinoma. In some embodiments, the perihilar bile duct cancer (also known as a hilar bile duct cancer) is squamous cell carcinoma.

In some embodiments, the method of treating a distal bile duct cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously. In some embodiments, the distal bile duct cancer is cholangiocarcinoma. In some embodiments, the distal bile duct cancer is adenocarcinoma. In some embodiments, the distal bile duct cancer is sarcoma. In some embodiments, the distal bile duct cancer is lymphoma. In some embodiments, the distal bile duct cancer is small-cell carcinoma. In some embodiments, the distal bile duct cancer is squamous cell carcinoma.

In some embodiments, the method of treating Klatskin tumor in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin. and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

In some embodiments, the method of treating a cholangiocarcinoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

In some embodiments, the method of treating an adenocarcinoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

In some embodiments, the method of treating a sarcoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the other therapeutic agent is a therapeutic antibody. In some embodiments, the other therapeutic agent is administered intravenously.

In some embodiments, the method of treating a lymphoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

In some embodiments, the method of treating a small-cell carcinoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-paclitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

In some embodiments, the method of treating a squamous cell carcinoma biliary tract cancer in an individual (e.g., human) comprises administering to the individual an effective amount of: a) a composition comprising nanoparticles comprising a taxane and an albumin, and b) another therapeutic agent. In some embodiments, the method comprises administering to the individual an effective amount of: a) a composition comprising nanoparticles comprising paclitaxel and an albumin, and b) another therapeutic agent. In some embodiments, the method comprises administering to the individual an effective amount of: a) a composition comprising nanoparticles comprising a taxane and an albumin, wherein the taxane in the nanoparticles is associated (e.g., coated) with the albumin, and b) another therapeutic agent. In some embodiments, the method comprises administering to the individual an effective amount of: a) a composition comprising nanoparticles comprising a taxane and an albumin, wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the nanoparticles comprise a taxane associated (e.g., coated) with albumin, and wherein the nanoparticles have an average particle size of no greater than about 200 nm, and b) another therapeutic agent. In some embodiments, the method comprises administering to the individual an effective amount of: a) a composition comprising nanoparticles comprising a taxane and an albumin, wherein the weight ratio of albumin and taxane in the nanoparticle composition is about 1:1 to about 9:1 (such as about 9:1), and b) another therapeutic agent. In some embodiments, the method comprises administering to the individual an effective amount of: a) a composition comprising nanoparticles comprising paclitaxel and human albumin, wherein the nanoparticles comprise paclitaxel associated (e.g., coated) with human albumin, wherein the nanoparticles have an average particle size of no greater than about 200 nm, and wherein the weight ratio of human albumin and paclitaxel in the nanoparticle composition is about 1:1 to about 9:1 (such as about 9:1), and b) another therapeutic agent. In some embodiments, the nanoparticle composition comprises nab-paclitaxel. In some embodiments, the nanoparticle composition is nab-pacitaxel. In some embodiments, the nanoparticle composition is administered at a dose of about 100-300 mg/m². In some embodiments, the nanoparticle composition is administered intravenously. In some embodiments, the other therapeutic agent is gemcitabine. In some embodiments, the other therapeutic agent is cisplatin. In some embodiments, the nanoparticle composition is administered in conjunction with an antimetabolite, such as gemcitabine, and a platinum-based agent, such as cisplatin. In some embodiments, the other therapeutic agent is a therapeutic antibody. In some embodiments, the other therapeutic agent is administered intravenously.

The dosing regimens for the methods described herein are further provided below.

Dosing and method of Administering the Nanoparticle Compositions

The dose of the taxane nanoparticle compositions administered to an individual (such as a human) may vary with the particular composition, the mode of administration, and the type of biliary tract cancer being treated. In some embodiments, the amount of the nanoparticle composition is effective to result in an objective response (such as a partial response or a complete response). In some embodiments, the amount of the taxane nanoparticle composition is sufficient to result in a complete response in the individual. In some embodiments, the amount of the taxane nanoparticle composition is sufficient to result in a partial response in the individual. In some embodiments, the amount of the taxane nanoparticle composition administered (for example when administered alone) is sufficient to produce an overall response rate of more than about any of 40%, 50%, 60%, or 64% among a population of individuals treated with the taxane nanoparticle composition. Responses of an individual to the treatment of the methods described herein can be determined, for example, based on RECIST levels.

In some embodiments, the amount of the nanoparticle composition is sufficient to prolong progress-free survival of the individual. In some embodiments, the amount of the nanoparticle composition is sufficient to prolong overall survival of the individual. In some embodiments, the amount of the nanoparticle composition (for example when administered along) is sufficient to produce clinical benefit of more than about any of 50%, 60%, 70%, or 77% among a population of individuals treated with the taxane nanoparticle composition.

In some embodiments, the amount of the nanoparticle composition, first therapy, second therapy, first-line treatment, second-line treatment, or combination therapy is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of biliary tract cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the taxane (e.g., paclitaxel) in the nanoparticle composition is below the level that induces a toxicological effect (i.e., an effect above a clinically acceptable level of toxicity) or is at a level where a potential side effect can be controlled or tolerated when the nanoparticle composition is administered to the individual.

In some embodiments, the amount of the nanoparticle composition is close to a maximum tolerated dose (MTD) of the nanoparticle composition following the same dosing regimen. In some embodiments, the amount of the nanoparticle composition is more than about any of 80%, 90%, 95%, or 98% of the MTD.

In some embodiments, the amount of a taxane (e.g., paclitaxel) in the nanoparticle composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of a taxane (e.g., paclitaxel) in the effective amount of the nanoparticle composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the nanoparticle composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of a taxane (e.g., paclitaxel) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m². 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a taxane (e.g., paclitaxel). In various embodiments, the nanoparticle composition includes less than about any of 350 mg/m². 300 mg/m². 250 mg/m², 200 mg/m², 150 mg/m², 120 mg/m², 100 mg/m², 90 mg/m², 50 mg/m², or 30 mg/m² of a taxane (e.g., paclitaxel). In some embodiments, the amount of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of a taxane (e.g., paclitaxel) in the nanoparticle composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of a taxane (e.g., paclitaxel) in the nanoparticle composition is about 5 to about 300 mg/m², such as about 100 to about 150 mg/m², about 120 mg/m², about 130 mg/m², or about 140 mg/m².

In some embodiments of any of the above aspects, the effective amount of a taxane (e.g., paclitaxel) in the nanoparticle composition includes at least about any of 1 mg/kg, 2.5 mg/kg, 3.5 mg/kg, 5 mg/kg, 6.5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, or 60 mg/kg. In various embodiments, the effective amount of a taxane (e.g., paclitaxel) in the nanoparticle composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

Exemplary dosing frequencies for the administration of the nanoparticle compositions include, but are not limited to, daily, every two days, every three days, every four days, every five days, every six days, weekly without break, three out of four weeks, once every three weeks, once every two weeks, or two out of three weeks. In some embodiments, the nanoparticle composition is administered about once every 2 weeks, once every 3 weeks, once every 4 weeks, once every 6 weeks, or once every 8 weeks. In some embodiments, the nanoparticle composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the taxane (e.g., paclitaxel) is administered over a period of at least ten days, wherein the interval between each administration is no more than about two days, and wherein the dose of the taxane (e.g., paclitaxel) at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the nanoparticle composition can be extended over an extended period of time, such as from about a month up to about seven years. In some embodiments, the nanoparticle composition is administered over a period of at least about any of 2, 3, 4, 5, 6, 7, 8, 9, 10, 11, 12, 18, 24, 30, 36, 48, 60, 72, or 84 months.

In some embodiments, the dosage of a taxane (e.g., paclitaxel) in a nanoparticle composition can be in the range of 5-400 mg/m² when given on a 3 week schedule, or 5-250 mg/m² (such as 80-150 mg/m², for example 100-120 mg/m² or 100-125 mg/m²) when given on a weekly schedule. For example, the amount of a taxane (e.g., paclitaxel) is about 60 to about 300 mg/m² (e.g., about 260 mg/m²) on a three week schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 100 mg/m², weekly, 2 out of 3 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks: 60-175 mg/m², weekly, without break; 20-150 mg/m² twice a week; and 150-250 mg/m² twice a week. The dosing frequency of the nanoparticle composition may be adjusted over the course of the treatment based on the judgment of the administering physician.

In some embodiments, the individual is treated for at least about any of one, two, three, four, five, six, seven, eight, nine, or ten treatment cycles.

The nanoparticle compositions described herein allow infusion of the nanoparticle composition to an individual over an infusion time that is shorter than about 24 hours. For example, in some embodiments, the nanoparticle composition is administered over an infusion period of less than about any of 24 hours, 12 hours, 8 hours, 5 hours, 3 hours, 2 hours, 1 hour, 30 minutes, 20 minutes, or 10 minutes. In some embodiments, the nanoparticle composition is administered over an infusion period of about 30 minutes.

Other exemplary doses of the taxane (in some embodiments paclitaxel) in the nanoparticle composition include, but are not limited to, about any of 50 mg/m², 60 mg/m², 75 mg/m². 80 mg/m². 90 mg/m², 100 mg/m², 120 mg/m², 125 mg/m², 160 mg/m², 175 mg/m², 200 mg/m², 210 mg/m², 220 mg/m², 260 mg/m², and 300 mg/m². For example, the dosage of paclitaxel in a nanoparticle composition can be in the range of about 100-400 mg/m² when given on a 3 week schedule, or about 50-275 mg/m² when given on a weekly schedule.

The nanoparticle compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the nanoparticle composition may be used. In some embodiments, the nanoparticle composition is administered intravenously. In some embodiments, the nanoparticle composition is administered intraportally. In some embodiments, the nanoparticle composition is administered intraarterially. In some embodiments, the nanoparticle composition is administered intraperitoneally. In some embodiments, the nanoparticle composition is administered intrahepatically.

### Modes of Administration of Combination Treatments

The dosing regimens for a composition comprising nanoparticles comprising a taxane and an albumin described herein apply to both monotherapy and combination treatment settings. The modes of administration for combination therapy methods are further described below.

In some embodiments, the nanoparticle composition and the other therapeutic agent (including the specific chemotherapeutic agents described herein) are administered simultaneously. When the drugs are administered simultaneously, the drug in the nanoparticles and the other therapeutic agent may be contained in the same composition (e.g., a composition comprising both the nanoparticles and the other therapeutic agent) or in separate compositions (e.g., the nanoparticles are contained in one composition and the other therapeutic agent is contained in another composition).

In some embodiments, the nanoparticle composition and the other therapeutic agent are administered sequentially. Either the nanoparticle composition or the other therapeutic agent may be administered first. The nanoparticle composition and the other therapeutic agent are contained in separate compositions, which may be contained in the same or different packages.

In some embodiments, the administration of the nanoparticle composition and the other therapeutic agent are concurrent, i.e., the administration period of the nanoparticle composition and that of the other therapeutic agent overlap with each other. In some embodiments, the nanoparticle composition is administered for at least one cycle (for example, at least any of 2, 3, or 4 cycles) prior to the administration of the other therapeutic agent. In some embodiments, the other therapeutic agent is administered for at least any of one, two, three, or four weeks. In some embodiments, the administrations of the nanoparticle composition and the other therapeutic agent are initiated at about the same time (for example, within any one of 1, 2, 3, 4, 5, 6, or 7 days). In some embodiments, the administrations of the nanoparticle composition and the other therapeutic agent are terminated at about the same time (for example, within any one of 1, 2, 3, 4, 5, 6, or 7 days). In some embodiments, the administration of the other therapeutic agent continues (for example for about any one of 1, 2, 3, 4, 5, 6, 7, 8, 9, 10, 11, or 12 months) after the termination of the administration of the nanoparticle composition. In some embodiments, the administration of the other therapeutic agent is initiated after (for example after about any one of 1, 2, 3, 4, 5, 6, 7, 8, 9, 10, 11, or we months) the initiation of the administration of the nanoparticle composition. In some embodiments, the administrations of the nanoparticle composition and the other therapeutic agent are initiated and terminated at about the same time. In some embodiments, the administrations of the nanoparticle composition and the other therapeutic agent are initiated at about the same time and the administration of the other therapeutic agent continues (for example for about any one of 1, 2, 3, 4, 5, 6, 7, 8, 9, 10, 11, or 12 months) after the termination of the administration of the nanoparticle composition. In some embodiments, the administration of the nanoparticle composition and the other therapeutic agent stop at about the same time and the administration of the other therapeutic agent is initiated after (for example after about any one of 1, 2, 3, 4, 5, 6, 7, 8, 9, 10, 11, or we months) the initiation of the administration of the nanoparticle composition.

In some embodiments, the administration of the nanoparticle composition and the other therapeutic agent are non-concurrent. For example, in some embodiments, the administration of the nanoparticle composition is terminated before the other therapeutic agent is administered. In some embodiments, the administration of the other therapeutic agent is terminated before the nanoparticle composition is administered. The time period between these two non-concurrent administrations can range from about two to eight weeks, such as about four weeks.

The dosing frequency of the drug-containing nanoparticle composition and the other therapeutic agent may be adjusted over the course of the treatment, based on the judgment of the administering physician. When administered separately, the drug-containing nanoparticle composition and the other therapeutic agent can be administered at different dosing frequency or intervals. For example, the drug-containing nanoparticle composition can be administered weekly, while a chemotherapeutic agent can be administered more or less frequently. In some embodiments, sustained continuous release formulation of the drug-containing nanoparticle and/or chemotherapeutic agent may be used. Various formulations and devices for achieving sustained release are known in the art. A combination of the administration configurations described herein can also be used.

The nanoparticle composition and the other therapeutic agent can be administered using the same route of administration or different routes of administration. In some embodiments (for both simultaneous and sequential administrations), the taxane in the nanoparticle composition and the other therapeutic agent are administered at a predetermined ratio. For example, in some embodiments, the ratio by weight of the taxane in the nanoparticle composition and the other therapeutic agent is about 1 to 1. In some embodiments, the weight ratio may be between about 0.001 to about 1 and about 1000 to about 1, or between about 0.01 to about 1 and 100 to about 1. In some embodiments, the ratio by weight of the taxane in the nanoparticle composition and the other therapeutic agent is less than about any of 100:1, 50:1, 30:1, 10:1, 9:1, 8:1, 7:1, 6:1, 5:1, 4:1, 3:1, 2:1, and 1:1 In some embodiments, the ratio by weight of the taxane in the nanoparticle composition and the other therapeutic agent is more than about any of 1:1, 2:1, 3:1, 4:1, 5:1, 6:1, 7:1, 8:1, 9:1, 30:1, 50:1, 100:1. Other ratios are contemplated.

The doses required for the taxane and/or the other therapeutic agent may (but not necessarily) be lower than what is normally required when each agent is administered alone. Thus, in some embodiments, the subtherapeutic amount of the drug in the nanoparticle composition and/or the other therapeutic agent is administered. “Subtherapeutic amount” or “subtherapeutic level” refer to an amount that is less than the therapeutic amount, that is, less than the amount normally used when the drug in the nanoparticle composition and/or the other therapeutic agent are administered alone. The reduction may be reflected in terms of the amount administered at a given administration and/or the amount administered over a given period of time (reduced frequency).

In some embodiments, other chemotherapeutic agent is administered so as to allow reduction of the normal dose of the drug in the nanoparticle composition required to effect the same degree of treatment by at least about any of 5%, 10%, 20%, 30%, 50%, 60%, 70%, 80%, 90%, or more. In some embodiments, enough drug in the nanoparticle composition is administered so as to allow reduction of the normal dose of the other therapeutic agent required to effect the same degree of treatment by at least about any of 5%, 10%, 20%, 30%, 50%, 60%, 70%, 80%, 90%, or more.

In some embodiments, the dose of both the taxane in the nanoparticle composition and the other therapeutic agent are reduced as compared to the corresponding normal dose of each when administered alone. In some embodiments, both the taxane in the nanoparticle composition and the other therapeutic agent are administered at a subtherapeutic, i.e., reduced, level. In some embodiments, the dose of the nanoparticle composition and/or the other therapeutic agent is substantially less than the established maximum toxic dose (MTD). For example, the dose of the nanoparticle composition and/or the other therapeutic agent is less than about 50%, 40%, 30%, 20%, or 10% of the MTD.

A combination of the administration configurations described herein can be used. The combination therapy methods described herein may be performed alone or in conjunction with another therapy, such as chemotherapy, radiation therapy, surgery, hormone therapy, gene therapy, immunotherapy, chemoimmunotherapy, hepatic artery-based therapy, cryotherapy, ultrasound therapy, liver transplantation, local ablative therapy, radiofrequency ablation therapy, photodynamic therapy, and the like. Additionally, a person having a greater risk of developing a biliary tract cancer may receive treatments to inhibit and/or delay the development of the disease.

The other therapeutic agent described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. In some embodiments, the other therapeutic agent is administrated intravenously. In some embodiments, the nanoparticle composition is administered orally.

The dosing frequency of the other therapeutic agent can be the same or different from that of the nanoparticle composition. Exemplary frequencies are provided above. As further example, the other therapeutic agent can be administered three times a day, two times a day, daily, 6 times a week, 5 times a week, 4 times a week, 3 times a week, two times a week, weekly. In some embodiments, the other therapeutic agent is administered twice daily or three times daily. Exemplary amounts of the other therapeutic agent include, but are not limited to, any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. For example, the other therapeutic agent can be administered at a dose of about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg).

In some embodiments, the effective amount of taxane in the nanoparticle composition is between about 45 mg/m² to about 350 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 350 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 300 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 150 mg/m² to about 350 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 150 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 100 mg/m². In some embodiments, the effective amount of taxane in the nanoparticle composition is between about 170 mg/m² to about 200 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 200 mg/m² to about 350 mg/m² and the effective amount of the other therapeutic agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 260 mg/m². In some embodiments of any of the above methods, the effective amount of the other therapeutic agent is about 20-30 mg/kg, about 30-40 mg/kg, about 40-50 mg/kg, about 50-60 mg/kg, about 60-70 mg/kg, about 70-80 mg/kg, about 80-100 mg/kg, or about 100-120 mg/kg.

In some embodiments, the effective amount of taxane in the nanoparticle composition is between about 75 mg/m² to about 150 mg/m², including, for example, about 100 mg/m² and about 125 mg/m², and the effective amount of the other therapeutic agent is about 20 mg/m² to about 1000 mg/mg², including, for example, about 25 mg/m², about 100 mg/m², about 500 mg/m², about 800 mg/m², and about 100 mg/m². In some embodiments, the other therapeutic agent is administered at a dosage recited in an alternate measurement, for example, platinum-based agents may be administered based on area under the curve (AUC). In some embodiments, the effective amount of the other therapeutic agent is about AUC=2, about AUC=3, AUC=4. AUC=5, or AUC=6.

In some embodiments, the taxane nanoparticle composition is administered with two or more other therapeutic agents. In some embodiments, the effective amount of taxane in the taxane nanoparticle composition is between about 75 mg/m² to about 150 mg/m², including, for example, about 100 mg/m² and about 125 mg/m², the effective amount of the first other therapeutic agent is about 20 mg/m² to about 50 mg/mg²′ including, for example, about 25 mg/m², about 30 mg/m², about 35 mg/m², about 40 mg/m², and about 45 mg/m², and the effective amount of the second other therapeutic agent is about 750 mg/m² to about 1250 mg/mg², including, for example, about 800 mg/m², about 900 mg/m², about 1000 mg/m², about 1100 mg/m², and about 1200 mg/m². In some embodiments, the other therapeutic agent is administered at a dosage recited in an alternate measurement, for example, platinum-based agents may be administered based on area under the curve (AUC). In some embodiments, the effective amount of another therapeutic agent is about AUC=2, about AUC=3, AUC=4, AUC=5, or AUC=6.

In some embodiments, the appropriate doses of other therapeutic agents are approximately those already employed in clinical therapies wherein the other therapeutic agent are administered alone or in combination with other therapeutic agents.

### Nanoparticle Compositions

The nanoparticle compositions described herein comprise nanoparticles comprising (in various embodiments consisting essentially of) a taxane (such as paclitaxel) and an albumin (such as human serum albumin). Nanoparticles of poorly water soluble drugs (such as taxane) have been disclosed in, for example, U.S. Pat. Nos. 5,916,596; 6,506,405; 6,749,868, and 6,537,579 and also in U.S. Pat. Pub. Nos. 2005/0004002, 2006/0263434, and 2007/0082838; PCT Patent Application WO08/137148, each of which is incorporated by reference in their entirety.

In some embodiments, the nanoparticle composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the nanoparticles in the nanoparticle composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the nanoparticle composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the nanoparticle composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the albumin has sulfhydral groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the nanoparticle composition are crosslinked (for example crosslinked through one or more disulfide bonds).

In some embodiments, the nanoparticles comprise the taxane (such as paclitaxel) coated with an albumin (e.g., human serum albumin). In some embodiments, the nanoparticle composition comprises taxane in both nanoparticle and non-nanoparticle forms, wherein at least about any one of 50%, 60%, 70%, 80%, 90%, 95%, or 99% of the taxane in the nanoparticle composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane that is substantially free of polymeric materials (such as polymeric matrix).

In some embodiments, the nanoparticle composition comprises albumin in both nanoparticle and non-nanoparticle portions of the nanoparticle composition, wherein at least about any one of 50%, 60%, 70%, 80%, 90%, 95%, or 99% of the albumin in the nanoparticle composition are in non-nanoparticle portion of the nanoparticle composition.

In some embodiments, the weight ratio of albumin. e.g., human albumin, to the taxane in the nanoparticle composition is such that a sufficient amount of taxane binds to, or is transported by, the cell. While the weight ratio of albumin to taxane will have to be optimized for different albumin and taxane combinations, generally the weight ratio of albumin, e.g., human albumin, to taxane (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and the taxane in the nanoparticle composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1. In some embodiments, the weight ratio of albumin (such as human serum albumin) and taxane in the nanoparticle composition is about 18:1 or less, such as about 15:1 or less, for example about 10:1 or less. In some embodiments, the weight ratio of albumin (such as human serum albumin) and taxane in the nanoparticle composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of albumin and taxane in the nanoparticle portion of the nanoparticle composition is about any one of 1:2, 1:3, 1:4, 1:5, 1:10, 1:15, or less.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery. Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed. McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med Bull., 46, 379-99 (1999). Kragh-Hansen, Dan. Med Bull., 1441, 131-40 (1990), Curry et al., Nat. Struct. Biol., 5, 827-35 (1998). Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human serum albumin) in the nanoparticle composition generally serves as a carrier for the taxane, i.e., the albumin in the nanoparticle composition makes the taxane more readily suspendable in an aqueous medium or helps maintain the suspension as compared to compositions not comprising an albumin. This can avoid the use of toxic solvents (or surfactants) for solubilizing the taxane, and thereby can reduce one or more side effects of administration of the taxane into an individual (such as a human). Thus, in some embodiments, the nanoparticle composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the nanoparticle composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin in the nanoparticle composition described herein will vary depending on other components in the nanoparticle composition. In some embodiments, the nanoparticle composition comprises an albumin in an amount that is sufficient to stabilize the taxane in an aqueous suspension, for example, in the form of a stable colloidal suspension (such as a stable suspension of nanoparticles). In some embodiments, the albumin is in an amount that reduces the sedimentation rate of the taxane in an aqueous medium. For particle-containing compositions, the amount of the albumin also depends on the size and density of nanoparticles of the taxane.

A taxane is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° ( ) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the taxane in an aqueous suspension at a certain concentration. For example, the concentration of the taxane in the nanoparticle composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the nanoparticle composition is free or substantially free of surfactant (such as Cremophor).

In some embodiments, the nanoparticle composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the nanoparticle composition, in liquid form, comprises about 0.5% to about 5% (w/v) of albumin.

In some embodiments, the albumin allows the nanoparticle composition to be administered to an individual (such as human) without significant side effects. In some embodiments, the albumin (such as human serum albumin) is in an amount that is effective to reduce one or more side effects of administration of the taxane to a human. The term “reducing one or more side effects of administration of the taxane” refers to reduction, alleviation, elimination, or avoidance of one or more undesirable effects caused by the taxane, as well as side effects caused by delivery vehicles (such as solvents that render the taxanes suitable for injection) used to deliver the taxane. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxanes can be reduced.

In some embodiments, the nanoparticle composition comprises Abraxane® (Nab-paclitaxel). In some embodiments, the nanoparticle composition is Abraxane® (Nab-paclitaxel). Abraxane® is a formulation of paclitaxel stabilized by human albumin USP, which can be dispersed in directly injectable physiological solution. When dispersed in a suitable aqueous medium such as 0.9% sodium chloride injection or 5% dextrose injection, Abraxane® forms a stable colloidal suspension of paclitaxel. The mean particle size of the nanoparticles in the colloidal suspension is about 130 nanometers. Since HSA is freely soluble in water, Abraxane® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle compositions are known in the art. For example, nanoparticles containing taxanes (such as paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868, and 6,537,579 and also in U.S. Pat. Pub. No. 2005/0004002, 2007/0082838, 2006/0263434 and PCT Application WO08/137148.

Briefly, the taxane (such as paclitaxel) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1.8:1, or 9:1.

### Other Components in the Nanoparticle Composition

The nanoparticles described herein can be present in a composition that includes other therapeutic agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine, stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the nanoparticle composition is suitable for administration to a human. In some embodiments, the nanoparticle composition is suitable for administration to a mammal such as, in the veterinary context, domestic pets and agricultural animals. There are a wide variety of suitable formulations of the nanoparticle composition (see, e.g., U.S. Pat. Nos. 5,916,596 and 6,096,331). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the nanoparticle composition is formulated to have a pH range of about 4.5 to about 9.0, including for example pH ranges of any of about 5.0 to about 8.0, about 6.5 to about 7.5, and about 6.5 to about 7.0. In some embodiments, the pH of the nanoparticle composition is formulated to no less than about 6, including for example no less than about any of 6.5, 7, or 8 (such as about 8). The nanoparticle composition can also be made to be isotonic with blood by the addition of a suitable tonicity modifier, such as glycerol.

### Kits, Medicines, Compositions, and Unit Dosages

The invention also provides kits, medicines, compositions, and unit dosage forms for use in any of the methods described herein.

Kits of the invention include one or more containers comprising taxane-containing nanoparticle compositions (or unit dosage forms and/or articles of manufacture) and/or another therapeutic agent (such as the agents described herein), and in some embodiments, further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

For example, in some embodiments, the kit comprises a) a composition comprising nanoparticles comprising a taxane and an albumin (such as human serum albumin), and b) instructions for administering the nanoparticle composition for treatment of a biliary tract cancer. In some embodiments, the kit comprises an effective amount of a) a composition comprising nanoparticles comprising a taxane and an albumin (such as human serum albumin), b) another therapeutic agent, and c) instructions for administering the nanoparticle composition and the other therapeutic agent for treatment of a biliary tract cancer. The nanoparticles and the other therapeutic agents can be present in separate containers or in a single container. For example, the kit may comprise one distinct composition or two or more compositions wherein one composition comprises nanoparticles and one composition comprises another therapeutic agent.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the nanoparticle compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the taxane (such as taxane) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the taxane and pharmaceutical compositions and instructions for use and packaged in quantities sufficient for storage and use in pharmacies, for example, hospital pharmacies and compounding pharmacies.

Also provided are medicines, medicament, combinations, compositions, and unit dosage forms useful for the methods described herein. In some embodiments, there is provided a medicine (or composition) for use in treating a biliary tract cancer comprising nanoparticles comprising a taxane and an albumin (such as human serum albumin). In some embodiments, there is provided a medicine (or composition) for use in treating a biliary tract cancer comprising nanoparticles comprising a taxane and an albumin (such as human serum albumin), wherein the medicine (or composition) is further administered with another therapeutic agent. In some embodiments, there is provided a medicine (or composition) for use in treating a biliary tract cancer comprising nanoparticles comprising a taxane and an albumin (such as human serum albumin), wherein the medicine (or composition) is further administered with at least one other therapeutic agent. In some embodiments, there is provided use of a composition comprising nanoparticles comprising a taxane and an albumin in the manufacture of a medicament for a biliary tract cancer in an individual. In some embodiments, there is provided use of a composition comprising nanoparticles comprising a taxane and an albumin in the manufacture of a medicament for a biliary tract cancer in an individual, wherein the medicament is further administered with another therapeutic agent. In some embodiments, there is provided use of a composition comprising nanoparticles comprising a taxane and an albumin in the manufacture of a medicament for a biliary tract cancer in an individual, wherein the medicament is further administered with at least one other therapeutic agent. In some embodiments, there is provided use of: a) a composition comprising nanoparticles comprising a taxane and an albumin, and b) another therapeutic agent in the manufacture of a medicament combination for a biliary tract cancer in an individual. In some embodiments, there is provided a combination comprising: a) a composition comprising nanoparticles comprising a taxane and an albumin, and b) another therapeutic agent, for use in treating a biliary tract cancer in an individual in need thereof.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

This example demonstrates a multi-institutional clinical study of the safety and efficacy of the combination of nab-paclitaxel and gemcitabine as a first-line treatment of cholangiocarcinoma.

This study is designed as a single-arm, two-stage study for first-line treatment of patients with advanced or metastatic cholangiocarcinoma. In stage I, patients receive, by intravenous administration, the combination of nab-paclitaxel at 125 mg/m² followed by gemcitabine at 1000 mg/m² on days 1, 8, and 15 of a 28-day cycle. Administration continues until disease progression, development of unacceptable toxicity, until in the opinion of the investigator the patient is no longer benefiting from therapy, at the Sponsor's request, withdrawal of consent, or death. If a population of patients enrolled in Stage I show progression free survival (PFS) at 6 months following administration, the study will be expanded to a second population in Stage II, who receive the same administration scheme as disclosed in Stage 1. Patients receive premedication per institutional standards.

Patient eligibility criteria includes: (i) having advanced or metastatic cholangiocarcinoma with no prior systemic chemotherapy; (ii) radiographically measurable disease per RECIST v.1.1; (iii) may have undergone surgery, received previous radiation, or liver-directed therapies; (iv) age of greater than 18 years old: (v) ECOG PS 0-1; and (vi) Child-Pugh<8.

An initial evaluation is performed for each patient, including obtaining general information (e.g., sex, race, age) and an initial laboratory evaluation. The initial laboratory evaluation includes assessment of: (i) ECOG PS; (ii) tumor location (e.g., intrahepatic, perihilar, distal extrahepatic); (iii) extent of disease (e.g., locally advanced, metastatic); and CA 19-9 level. Patient blood and tumor samples are also collected prior to administration of the study treatment to allow for biomarker evaluation, including circulating tumor cells (CTCs), cytidine deaminase (CDA), human equilibrative nucleoside transporter 1 (hENT1), secreted protein acidic and rich in cysteine (SPARC), and fibrosis. Presence of gemcitabine (active and inactive) metabolites will also be assessed.

Study treatment begins within 10 working days of patient registration. During the course of treatment, patients receive radiographic assessment every 8 weeks, starting with the initial evaluation, to evaluate response to treatment via RECIST v1.1. Further patient blood and tumor samples may be collected during treatment. Patient blood and/or tumor samples may be collected following completion or discontinuation of treatment.

Adverse events are monitored during treatment, including monitoring of neutropenia, thrombocytopenia, fatigue, anemia, leukopenia, peripheral neuropathy, diarrhea, sepsis, hyponatremia, and increase in alanine aminotransferase (ALT). Adverse events are monitored by, e.g., physical examination, vital signs, ECG, and laboratory assessments (e.g., serum chemistry, hematology).

The primary endpoint is progression free survival (PFS) at 6 months following administration. Secondary endpoints include safety, median time to progression (TTP), overall response rate (ORR), disease control rate (DCR), median progression free survival (PFS), median overall survival (OS), and correlation of change in CA 19-9 to clinical efficacy. Exploratory objectives include correlating changes in CTCs with survival measurements and correlating CDA, hENT1, SPARC, including stromal CDA, hENT1, and SPARC, with survival measurements.

### Example 2

This example demonstrates a multi-institutional clinical study of the safety and efficacy of the combination of nab-paclitaxel, gemcitabine, and cisplatin for treatment of biliary tract cancer.

This study is designed as a single-arm, two dosage group study for first-line treatment of patients with biliary tract cancer. All patients are administered, intravenously, nab-paclitaxel followed by cisplatin and, subsequently, gemcitabine, on days 1 and 8 or a 21-day cycle. Patients in the higher dosage group are initially treated with 125 mg/m² nab-paclitaxel, 1000 mg/m² gemcitabine, and 25 mg/m² cisplatin. Patients in the lower dosage group are initially treated with 100 mg/m² nab-paclitaxel, 800 mg/m² gemcitabine, and 25 mg/m² cisplatin. Dose modifications, e.g., reductions, interruptions, and growth factor treatment are permitted for treatment-related toxicity. Administration continues until disease progression, development of unacceptable toxicity, until in the opinion of the investigator the patient is no longer benefiting from therapy, at the Sponsor's request, withdrawal of consent, or death.

Patient inclusion eligibility criteria includes: (i) being greater than or 18 years old; (ii) histologically or cytologically confirmed intra- or extrahepatic cholangiocarcinoma or gallbladder cancer, (iii) metastatic or unresectable disease documented on diagnostic imagining studies; (iv) no prior chemotherapy (prior adjuvant therapy is permitted provided that it was received greater than 6 months before the first does of trial medication; (v) ECOG PS≤1; and (vi) adequate hematologic, hepatic, and renal function.

Patient exclusion criteria includes: (i) peripheral neuropathy of grade≥2: (ii) concurrent severe and/or uncontrolled medical conditions that could compromise trial participation; (iii) pregnancy or lactation in females; and (iv) known central nervous system disease (with the exception of treated brain metastasis.

An initial evaluation is performed for each patient, including general information, e.g., sex, race, age, and an initial laboratory evaluation. The initial laboratory evaluation includes assessment of: (i) ECOG PS; (ii) tumor location and type (e.g., extrahepatic cholangiocarcinoma, intrahepatic cholangiocarcinoma, gallbladder cancer): (iii) disease stage (e.g., locally advanced, metastatic); and CA 19-9 level.

Response to treatment is assessed about every 3 cycles according to RECIST criteria.

Adverse events are monitored during treatment, including monitoring of neutropenia, thrombocytopenia, fatigue, anemia, leukopenia, peripheral neuropathy, diarrhea, sepsis, hyponatremia, and increase in alanine aminotransferase (ALT). Adverse events are monitored by, e.g., physical examination, vital signs, ECG, laboratory assessments (e.g., serum chemistry, hematology).

The primary endpoint is progression free survival (PFS). Secondary endpoint objectives include response rate (RR), disease control rate (DCR; defined as partial response (PR) plus complete response (CR) plus stable disease (SD) rate), overall survival (OS), and safety.

1: A method of treating a biliary tract cancer in an individual in need thereof, comprising administering to the individual an effective amount of a composition comprising nanoparticles comprising a taxane and an albumin.

2: The method of claim 1, wherein the biliary tract cancer is an intrahepatic bile duct cancer.

3: The method of claim 1, wherein the biliary tract cancer is an extrahepatic bile duct cancer.

4: The method of claim 3, wherein the extrahepatic bile duct cancer is a perihilar bile duct cancer or a distal bile duct cancer.

5: The method of claim 3, wherein the extrahepatic bile duct cancer is Klatskin tumor.

6: The method of claim 1, wherein the biliary tract cancer is cholangiocarcinoma.

7: The method of claim 1, wherein the biliary tract cancer is adenocarcinoma.

8: The method of claim 1, wherein the biliary tract cancer is sarcoma, lymphoma, small-cell carcinoma, or squamous cell carcinoma.

9: The method of claim 1, wherein the biliary tract cancer is early stage biliary tract cancer, non-metastatic biliary tract cancer, primary biliary tract cancer, advanced biliary tract cancer, locally advanced biliary tract cancer, metastatic biliary tract cancer, biliary tract cancer in remission, recurrent biliary tract cancer, biliary tract cancer in an adjuvant setting, or biliary tract cancer in a neoadjuvant setting.

10: The method of claim 1, further comprising administering another therapeutic agent.

11: The method of claim 10, wherein the nanoparticle composition and the other therapeutic agent are administered simultaneously or sequentially.

12: The method of claim 10, wherein the nanoparticle composition and the other therapeutic agent are administered concurrently.

13: The method of claim 1, wherein the composition comprising nanoparticles comprising taxane and albumin is administered intravenously, intraarterially, intraperitoneally, intravesicularly, subcutaneously, intrathecally, intrapulmonarily, intramuscularly, intratracheally, intraocularly, transdermally, intradermally, orally, intraportally, intrahepatically, hepatic arterial infusion, or by inhalation.

14: The method of claim 13, wherein the composition comprising nanoparticles comprising a taxane and albumin is administered intravenously, intraarterially, intrahepatically, or intraportally.

15: The method of claim 10, wherein the other therapeutic agent is administered intravenously.

16: The method of claim 1, wherein the taxane is paclitaxel.

17: The method of claim 1, wherein the nanoparticles in the composition have an average diameter of no greater than about 200 nm.

18: The method of claim 1, wherein the taxane in the nanoparticles is coated with albumin.

19: The method of claim 1, wherein the weight ratio of albumin and taxane in the nanoparticle composition is about 1:1 to about 9:1.

20: The method of claim 19, wherein the weight ratio of albumin and taxane in the nanoparticle composition is about 9:1.

21: The method of claim 1, wherein the albumin is human albumin.

22: The method of claim 1, wherein the albumin is human serum albumin.

23: The method of claim 1, wherein the individual is human.

24: A kit comprising: a) a composition comprising nanoparticles comprising a taxane and an albumin, and b) an instruction for using the nanoparticle composition for treating a biliary tract cancer in an individual.

25: The kit of claim 24, further comprising another therapeutic agent.
